(19) United States
(12) Patent Application Publication
(10) Pub. No.: US 2024/0278172 A1
(43) Pub. Date: Aug. 22, 2024

(54) ELECTROSPUN POLYMERIC NANOFIBER FILTER MATERIAL AND DEVICES

(71) Applicant: Liquidity Corporation, Concord, CA (US)

(72) Inventors: Tomás Colussi, Oakland, CA (US); Ashwin Gulati, Concord, CA (US)

(73) Assignee: Liquidity Corporation, Concord, CA (US)

(21) Appl. No.: 18/653,657

(22) Filed: May 2, 2024

Related U.S. Application Data
(63) Continuation of application No. PCT/US2022/048825, filed on Nov. 3, 2022.
(60) Provisional application No. 63/304,933, filed on Jan. 31, 2022.
(60) Provisional application No. 63/275,069, filed on Nov. 3, 2021.
(60) Provisional application No. 63/275,086, filed on Nov. 3, 2021.
(60) Provisional application No. 63/275,101, filed on Nov. 3, 2021.

(51) Int. Cl. B01D 53/22; A47B 96/20; B01D 39/16; B01D 46/00; B01D 46/54; B01D 63/14; B01D 69/02; B01D 69/10; B01D 71/06; F24F 8/108

(52) U.S. Cl. CPC B01D 53/228; B01D 39/163; B01D 46/0028; B01D 46/543; B01D 63/14; B01D 69/02; B01D 69/1071; B01D 71/06; F24F 8/108; A47B 96/20; B01D 2053/221; B01D 2239/025; B01D 2239/0442; B01D 2239/0492; B01D 2239/0631; B01D 2239/1233; B01D 2239/1258; B01D 2279/50; B01D 2279/65; B01D 2313/44; B01D 2325/04; B01D 2325/20; B01D 2325/44; B01D 2325/48

(57) ABSTRACT

An air filtration membrane includes a substrate and a network of electrospun polymeric nanofibers deposited on the substrate, wherein the air filtration membrane has a minimum air filtration efficiency of between MERV 13 and HEPA filtration. One or more layers of the air filtration membrane can include applied antimicrobial nanoparticles.

### CROSS-REFERENCE TO RELATED APPLICATIONS

This application is a continuation of International Patent Application No. PCT/US2022/048825 filed Nov. 3, 2022, which claims priority to and the benefit of U.S. Provisional Application No. 63/275,069 filed 3 Nov. 2021, U.S. Provisional Application No. 63/275,086 filed 3 Nov. 2021, U.S. Provisional Application. No. 63/275,101 filed 3 Nov. 2021, and U.S. Provisional Application No. 63/304,933 filed 31 Jan. 2022, each of which is incorporated herein by reference in its entirety.

### BACKGROUND OF THE INVENTION

### 1. Field of the Invention

The subject invention is directed to air filtration media, and more particularly, to air filters for HVAC applications, window screens, indoor barrier screens or partitions, and modular furniture that use an electro-spun polymeric nanofiber membrane media with high filtering efficiency and excellent porosity for air filtration, and which optionally include antimicrobial compounds incorporated therewith.

### 2. Description of Related Art

Gaseous flows (e.g. air flows) often carry undesirable entrained particulates. Filters are commonly employed to remove some or all of the particulates from the gaseous flows. Filter media including nanofibers formed using an electrostatic spinning process is also known as disclosed for example, in U.S. Pat. No. 8,172,092, the disclosure of which is incorporated herein by reference. Nanoparticle compositions used for making antimicrobial or antiviral agents to treat fibrous materials are also known, as disclosed for example in U.S. Pat. No. 9,434,006, the disclosure of which is incorporated herein by reference.

Applicant recognizes that it would be beneficial to provide electrospun polymer nanofibrous membranes that provide high filtering efficiency and excellent porosity, optionally wherein the membrane is treated with one or more antimicrobial or antiviral agents. Such a membrane can be particularly useful in blocking and inactivating pathogens, including viruses such as COVID-19.

The subject electrospun polymeric nanofiber membranes are suitable for use in indoor air filtration applications, such as in heating, ventilation and air conditioning (HVAC) systems, as well as window screens, furniture such as room dividers, movable or stationary indoor barrier screens, and modular furniture panels, such as cubicle panels.

### SUMMARY OF THE INVENTION

In accordance with one aspect of the invention, an air filtration membrane is provided, which includes a substrate and a network of electrospun polymeric nanofibers deposited on the substrate, wherein the network has a total polymer weight of 5 to 25% and wherein said air filtration membrane has a minimum air filtration efficiency of between MERV 13 and HEPA filtration.

The polymeric nanofibers can include polyacrylonitrile, polyvinylidene fluoride, thermoplastic polyurethane or polypropylene. The polymeric nanofibers can have an average diameter of 50 to 500 nm. The polymeric nanofibers can create a layer of thickness between 0 to 150 microns. The substrate material can include non-woven polyester fibrous or microfibrous material, or non-woven polypropylene fibrous material.

The substrate material can include a polyester screen with mesh openings ranging from 50 to 1000 microns. The substrate material can include a vinyl coated polyester screen. The substrate material can include a vinyl coated fiberglass screen.

Either the polymeric nanofibers or substrate can be coated with an antifungal, antibacterial, and/or antiviral agent. Either the polymeric nanofibers or substrate can be treated with non-spherical, spherical or coral shaped metal nanoparticles, on the surface thereof.

The air filtration membrane can have a visible light transmittance range from 0 to 60%. The air filtration membrane can have a minimum Frazier air permeability of about 10 L/m²/s at 125 Pa.

The air filtration membrane can have a protective layer that is thermally laminated or bonded to the membrane for structural support. The membrane can be pleated to maximize filtration surface area.

The network of polymeric nanofibers can include multiple polymer types, which include multiple layers of different nanofibers, or multiple polymers co-spun together to form a blended layer.

The air filtration membrane can be incorporated into a filter for use in HVAC systems. The air filtration membrane can be incorporated into a window screen. The air filtration membrane can be incorporated into indoor barrier screens.

The air filtration membrane can be incorporated into indoor modular furniture panels.

The membrane can be configured as a replaceable filter cartridge adapted to slide vertically or horizontally into the modular furniture panel frame and locked into place therein.

The membrane can include one or both sides of a lightweight, permeable fabric sleeve configured to cover a modular furniture panel frame.

The modular furniture panels can be adapted and configured to be attached together by way of brackets or clamps. The brackets or clamps can be magnetized for ease of assembly. The air filtration membrane can have high air permeability.

In accordance with another aspect of the invention, a modular furniture panel, is provided, which includes a structural frame, and an air filtration membrane operatively associated with the frame, wherein the membrane includes a substrate and a network of electro-spun polymeric nanofibers deposited on the substrate.

The network can have a total polymer weight of 5 to 25% and the membrane can have a minimum air filtration efficiency of between MERV 8 and MERV 16. The membrane can be configured as a filter cartridge adapted for insertion into the frame of the modular furniture panel. The membrane can be configured as an air permeable sleeve adapted to enclose the frame of the modular furniture panel. The frame can include means for connecting plural modular furniture panels together to construct an assembly of furniture panels.

### BRIEF DESCRIPTION OF THE DRAWINGS

So that those skilled in the art will readily understand how to make and use the air filtration devices of the subject invention without undue experimentation, preferred embodiments thereof will be described in detail hereinbelow with reference to the figures wherein:

FIG. 1 is a top view electron scanning microscope image of one embodiment of an electrospun nanofiber air filter membrane in accordance with the present invention;

FIG. 2 is an end view electron scanning microscope image of an electrospun nanofiber air filter membrane in accordance with another aspect of the invention, wherein antimicrobial nanoparticles have been applied to the surface thereof;

FIG. 3 is an illustration of a process for making electrospun nanofiber membranes in accordance with the present invention;

FIG. 4 is an illustration of an indoor environment in which the air filtration devices of the present invention are incorporated into window screens, barrier screens, and in air conditioning and/or mechanical ventilation systems;

FIG. 5 is an illustration of an HVAC air filter that utilizes an electrospun nanofiber membrane in accordance with the present invention;

FIG. 6 is an illustration of a window screen that utilizes an electrospun nanofiber membrane in accordance with the present invention;

FIG. 7 is an illustration of an indoor barrier screen that utilizes an electrospun nanofiber membrane in accordance with the present invention;

FIG. 8 is an illustration of modular office panels utilizing an electrospun nanofiber membrane in accordance with the invention, joining together to form an individual workspace cubicle;

FIG. 9 is an illustration showing one embodiment of modular office panels in accordance with the invention, which utilize the subject electrospun nanofiber membrane materials; and

FIG. 10 is an illustration showing a second embodiment of modular office panels in accordance with the invention, which utilize the subject electrospun nanofiber membrane materials.

### DETAILED DESCRIPTION OF THE INVENTION

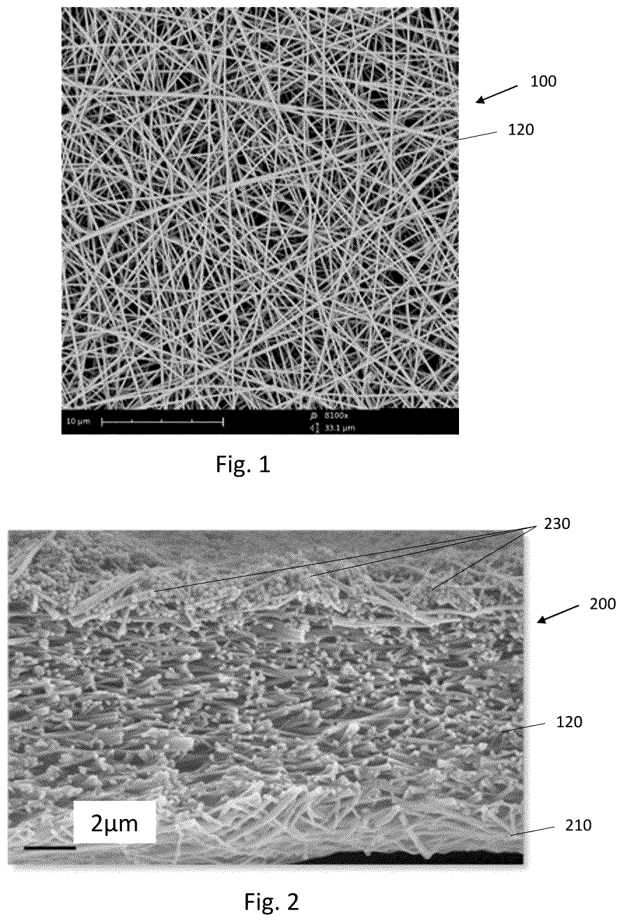

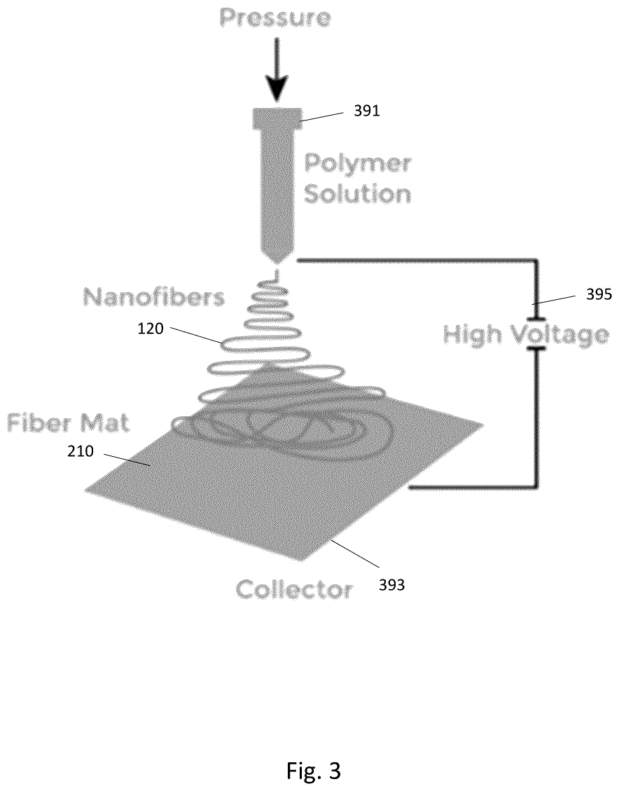

With reference to FIGS. 1-3, the subject invention is directed to air filtration membranes 100, 200 that include a substrate 210 and a network of electrospun polymeric nanofibers 120, deposited on the substrate 210. During the electrospinning process, which is illustrated in FIG. 3, thousands of nanofibers 120 are spun into a dense mesh, which has 60 to 80% open area, as compared to standard commercially available membranes that have 25% open area.

The nano-dimension of the nanofibers 120 gives the subject membranes 100, 200 a high surface area to volume ratio, making it very attractive in applications where high surface area and flow are desirable. Moreover, there is a relatively low pressure drop across the filtration membranes 100, 200, which saves energy when used in connection with mechanical ventilation systems.

The air filtration membranes 100, 200 of the subject invention preferably have a minimum air filtration efficiency of MERV 13, and can have air filtration efficiencies as high as MERV 16 and beyond, to HEPA quality filtration, depending on the implementation. In related terms, the subject air filtration membranes can filter particulate matter ranging from PM10 to PM1.0.

The polymeric nanofibers of the filtration membrane can comprise polyacrylonitrile (PAN), polyvinylidene fluoride (PVDF), thermoplastic polyurethane (TPU), polypropylene (PP), or a similar thermoplastic material. Preferably, the polymeric nanofibers in the filtration membrane have an average diameter of 50-500 nm and the membrane has a layer of thickness between 0-150 microns. If desired, the network of polymeric nanofibers can be composed of multiple polymer types (either multiple layers of different nanofibers, or multiple polymers co-spun together to form a blended layer).

For certain implementations, including HVAC applications, the substrate material can comprise non-woven polyester fibrous or microfibrous material, or non-woven polypropylene fibrous material. In other air filtration applications, the substrate material can comprise a polyester screen with mesh opening ranging from 50 to 1000 microns. More particularly, the substrate material can comprise a vinyl coated polyester screen or a vinyl coated fiberglass screen.

The polymeric nanofibers and/or the substrate can be coated with one or more antifungal, antibacterial, and/or antiviral agents. In a preferred aspect, the polymeric nanofibers and/or the substrate are sprayed or otherwise treated with non-spherical, spherical or coral shaped activated metal nanoparticles 230. As a result, such membranes can quickly block droplets carrying a range of viruses including COVID-19, and quickly inactivate pathogens through interaction with the aforementioned metal nanoparticles.

In accordance with the invention, the subject air filtration membranes preferably have a visible light transmittance range of 0-60% and a minimum Frazier air permeability of 10 L/m²/s at 125 Pa. Depending on the precise embodiment, a protective layer is preferably thermally laminated or otherwise bonded to the nanofiber membrane to provide additional structural support or protection.

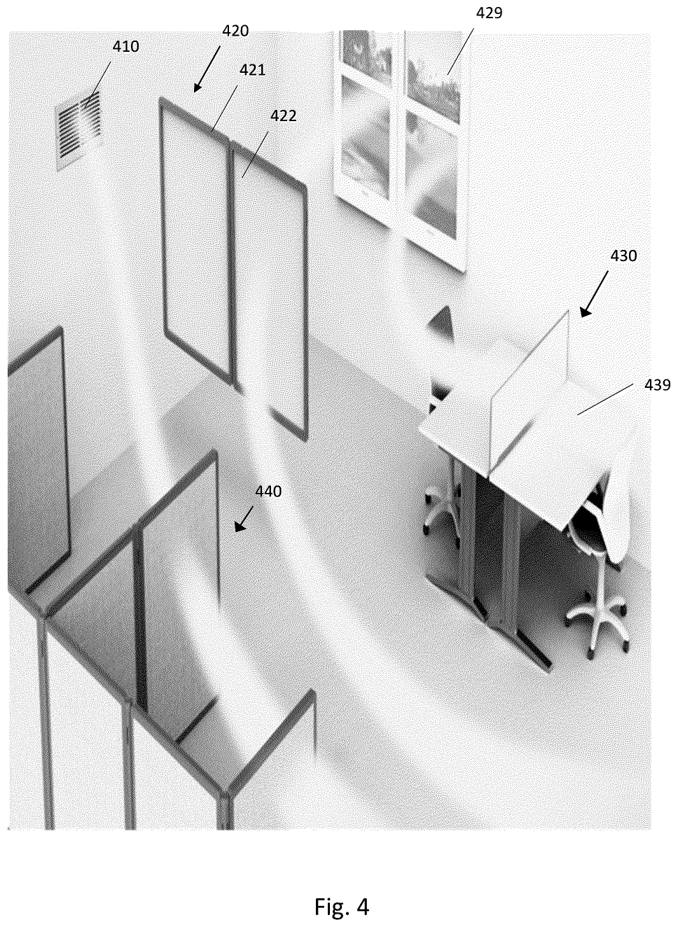

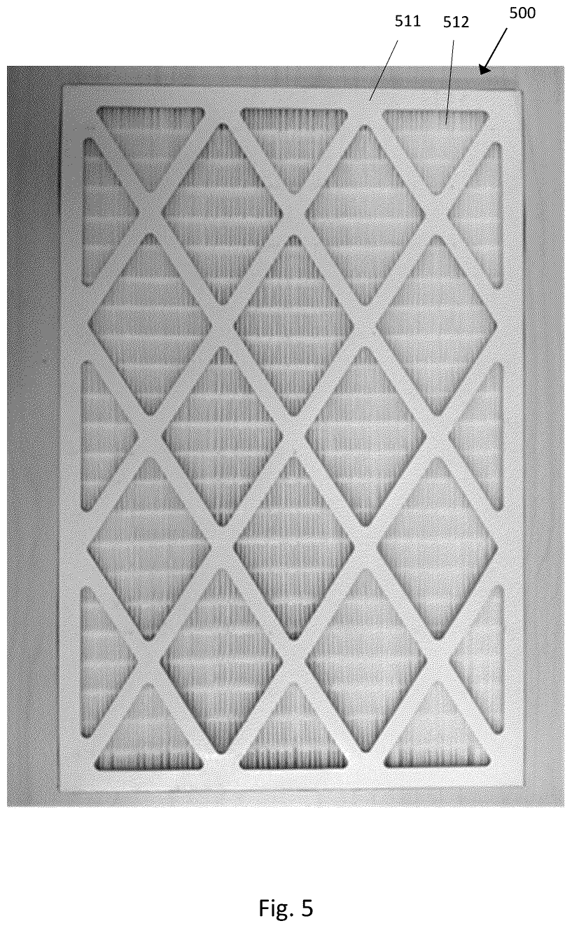

With reference to FIGS. 4-7, the subject air filtration membranes 100, 200 can be employed in a variety of applications within an indoor environment. For example, the subject air filtration membranes can be included in a framed 511 filter device 500 used in building air conditioning, heating or ventilation systems 410, as shown in FIGS. 4 and 5. In such applications, the filtration membrane can be pleated to maximize filtration surface area. Such air filter devices can be provided in a variety of standard sizes, for example 24 inches×24 inches, and a variety of standard thicknesses, for example 1 inch to 4 inches. The subject air filtration membranes can further advantageously be employed in automotive and/or aerospace air filtration applications.

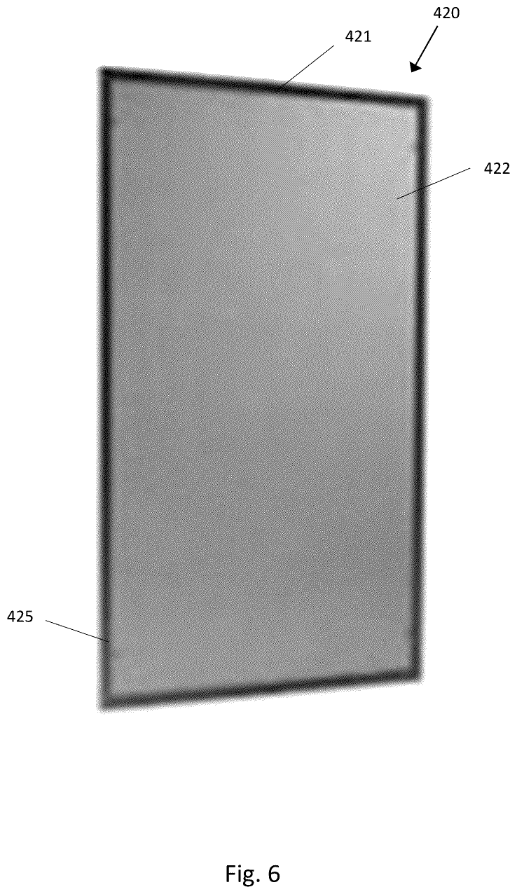

In accordance with a further aspect of the invention, the subject membranes can be incorporated into window screens 420 as shown in FIGS. 4 and 6. The membrane 422 for window screens can be provided in a structural frame 421 or it can be provided in media rolls. When provided in a structural frame 421, connectors 425 can be provided to engage with a window frame 429 or screen mounting area. Such filtering window screens enable ventilation during high Air Quality Index (AQI) days and are optimized for the best combination of light transmittance, air permeability and MERV 16 filtration. The screens are useful for filtering smog, pollen, dust, dirt, allergens, bacteria, mold, and airborne viruses (e.g.: SARS-COV-2), and they enhance existing HVAC air flow filtration.

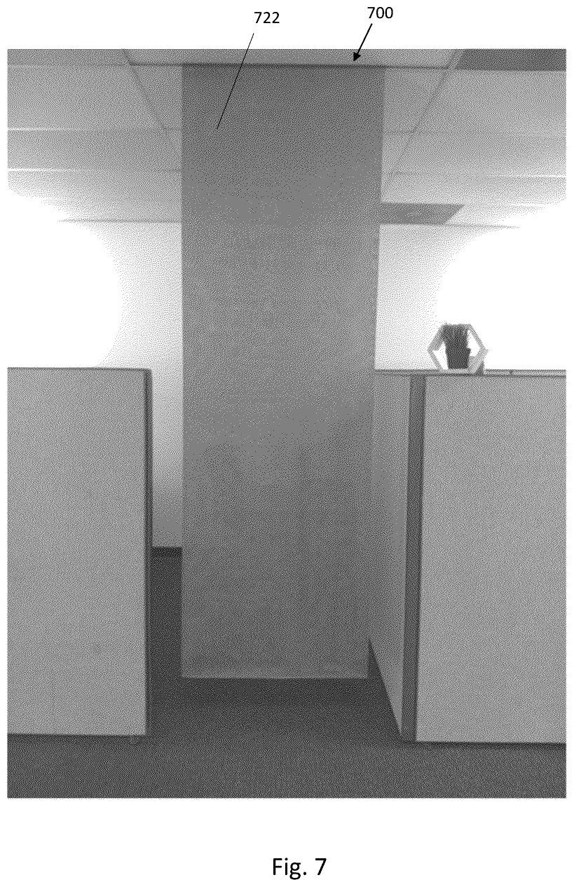

In accordance with still a further aspect of the invention, the subject membranes can be incorporated into indoor barrier screens 430, 440, 700 as shown in FIGS. 4 and 7. Assembled barriers screens can be provided so as to sit on a tabletop 439 or countertop, freestanding 440, or in a wall-mounted or hanging style, such as in barrier screen 700 of FIG. 7. Such hanging screens can optionally be mounted to a spring-wound or motorized reel to allow for deployment only when needed. Such barrier screens are preferably see-through, talk-through and puncture resistant.

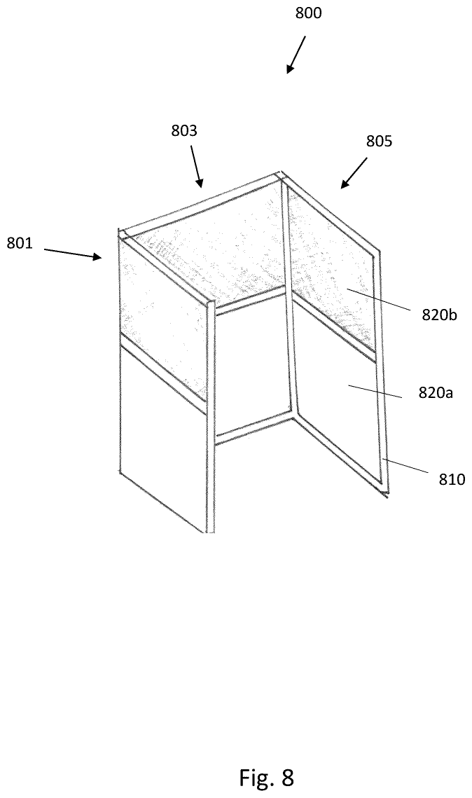

Referring now to FIGS. 8-10, in accordance with a further aspect of the present invention, the subject nanofiber filtration membranes are incorporated into or otherwise operatively associated with air permeable panels that are adapted and configured to be assembled together in a desired orientation.

In accordance with this aspect, the panels can be used as barrier walls or room dividers, or several panels of various sizes and/or shapes can be assembled together to construct modular office furniture or cubicles 800, as shown for example in FIG. 8.

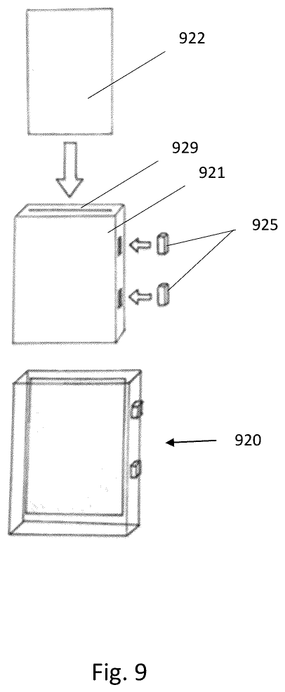

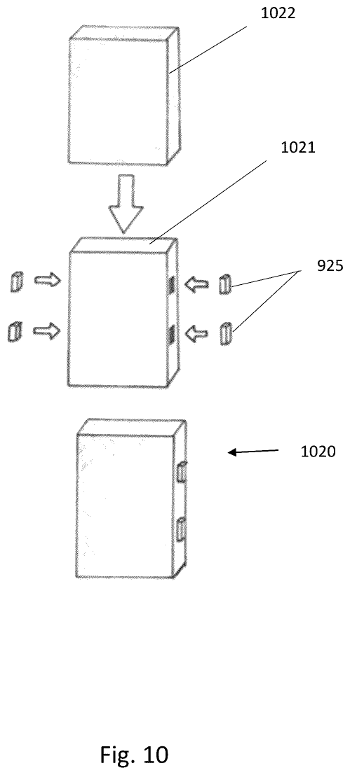

With reference to FIG. 8, three panels 801, 803, 805 are secured together using mechanical fasteners 925 such as clips, snaps or clamps, screws, pins or magnetic fasteners, depending on the desired implementation. Such fasteners 925 are illustrated in FIGS. 9 and 10.

As illustrated, the panels 801, 803, 805 are preferably constructed with a structural frame 810 that is made from a breathable material, i.e., a material formed with holes or openings so that air can flow through, such as mesh, fabric, metal or wood. Alternatively, solid frame materials can be used while still gaining many benefits of the subject nanofiber filtration membranes. In the embodiment of FIG. 8, each frame 810 holds one or more insert structures 820a, 820b, at least some of which include nanofiber filtration membranes in accordance with the invention. In the exemplary cubicle 800 of FIG. 8, upper panels 820b are formed from a nanofiber filter material in accordance with the invention, to filter air while allowing passive airflow therethrough, while the lower panels 820a are formed from a more conventional cubicle material, such as pressed fiberboard. Alternatively, both upper 820b and lower 820a panels can be made of a nanofiber filter material in accordance with the invention.

With reference to FIG. 9, one embodiment of an interior partition panel 920 is illustrated in which the nanofiber filtration membrane is configured as a filter cartridge 922 that can be readily inserted into an opening 929 formed in a panel 921 and locked or otherwise secured into place. In this manner, the filter cartridge 922 can be removed and replaced if needed, without needing to replace an entire panel 920. Such panels 920 can further be covered by decorative, air-permeable fabrics, if desired.

Depending on the implementation, the opening 929 can be in the top edge (illustrated), or alternatively, bottom edge or either side edge of the panel 921, so the cartridge 922 can be inserted into the panel 921 either vertically or horizontally.

As shown in FIG. 8, multiple panels 801, 803, 805 can be secured together to create a cubicle 800 or other configuration using mechanical fasteners 925 such as clips, snaps or clamps, screws, pins or magnetic fasteners, depending on the desired implementation. Alternatively, strong hook-and-loop fasteners can be employed in other embodiments, as can be permanent or temporary adhesives.

With reference to FIG. 10, another embodiment of interior partition panel 1020 is illustrated, in which the filtration membrane is configured as an air permeable sleeve 1022 or “sock” that is adapted to cover over or otherwise encapsulate and enclose the panel body 1021 and frame, if provided.

In one application, the panel body 1021 and/or frame are uniquely fabricated for this purpose. In alternative applications, the body 1021, just frame, or both are reclaimed from an existing piece of modular office furniture and retrofit by covering with the filtration membrane sleeve 1022 or inserting a filter cartridge 922 into a reclaimed frame.

In accordance with one aspect of the invention, the air permeable fabric used to construct the sleeve 1022 can be coated with antimicrobial metal oxide particles such as Titanium Dioxide (TiO₂) in order to promote photocatalytic inactivation of microorganisms in the presence of UV light. Such particles can be formed directly into the fabric, or alternatively applied to the outer surface thereof by spraying or other suitable method.

The filtration membrane elements, such as cartridge 922, and sleeve 1022, employed in the modular furniture panels described and illustrated herein preferably include a substrate and a network of electrospun polymeric nanofibers deposited on the substrate. The network of electrospun polymeric nanofibers has a total polymer weight of 5 to 25% and the membrane has a minimum air filtration efficiency of between MERV 8 and MERV 16. Particularly, the membrane can have a minimum air filtration efficiency of MERV 8, MERV 9, MERV 10, MERV 11, MERV 12, MERV 13, MERV 14, MERV 15 or MERV 16.

In accordance with this aspect, the substrate material can comprise non-woven polyester fibrous or microfibrous material and/or a non-woven polypropylene fibrous material. Additionally or alternatively, the substrate material can comprise a polyester screen with mesh openings ranging from 50 to 1000 microns. The polymeric nanofibers can comprise polyacrylonitrile (PAN), polyvinylidene fluoride (PVDF), thermoplastic polyurethane (TPU), polypropylene (PP), or a similar thermoplastic material, have an average diameter of 50 to 500 nm, and create a layer of thickness of between 0 to 150 microns.

In accordance with this aspect, the polymeric nanofibers and/or the substrate can be coated with an antifungal, antibacterial, and/or antiviral agent. In such an instance, either the polymeric nanofibers and/or the substrate can be sprayed with non-spherical, spherical, or coral shaped metal nanoparticles.

Moreover, a protective layer can laminated or bonded to the membrane for structural support and the network of polymeric nanofibers can include multiple polymer types, which include multiple layers of different nanofibers, or multiple polymers co-spun together to form a blended layer.

While the subject disclosure has been shown and described with reference to preferred embodiments, those skilled in the art will readily appreciate that changes and/or modifications may be made thereto without departing from the spirit or scope of the present invention.

What is claimed is:

1. An air filtration membrane comprising:
a substrate; and
a network of electrospun polymeric nanofibers deposited on the substrate, wherein the network has a total polymer weight of 5 to 25% and wherein said air filtration membrane has a minimum air filtration efficiency of between MERV 13 and HEPA filtration.

2. The air filtration membrane of claim 1, wherein the polymeric nanofibers comprise polyacrylonitrile, polyvinylidene fluoride, thermoplastic polyurethane or polypropylene.

3. The air filtration membrane of claim 1, wherein the polymeric nanofibers have an average diameter of 50 to 500 nm.

4. The air filtration membrane of claim 1, wherein the polymeric nanofibers create a layer of thickness between 0 to 150 microns.

5. The air filtration membrane of claim 1, wherein the substrate material comprises non-woven polyester fibrous or microfibrous material, or non-woven polypropylene fibrous material.

6. The air filtration membrane of claim 1, wherein the substrate material comprises a polyester screen with mesh openings ranging from 50 to 1000 microns.

7. The air filtration membrane of claim 1, wherein the substrate material comprises a vinyl coated polyester screen.

8. The air filtration membrane of claim 1, wherein the substrate material comprises a vinyl coated fiberglass screen.

9. The air filtration membrane of claim 1, wherein either the polymeric nanofibers or substrate is coated with an antifungal, antibacterial, and/or antiviral agent.

10. The air filtration membrane of claim 1, wherein either the polymeric nanofibers or substrate is treated with non-spherical, spherical or coral shaped metal nanoparticles, on the surface thereof.

11. The air filtration membrane of claim 1, having a visible light transmittance range from 0 to 60%.

12. The air filtration membrane of claim 1, having a minimum Frazier air permeability of about 10 L/m²/s at 125 Pa.

13. The air filtration membrane of claim 1, wherein a protective layer is thermally laminated or bonded to the membrane for structural support.

14. The air filtration membrane of claim 1, wherein the membrane is pleated to maximize filtration surface area.

15. The air filtration membrane of claim 1, wherein the network of polymeric nanofibers includes multiple polymer types, which include multiple layers of different nanofibers, or multiple polymers co-spun together to form a blended layer.

16. The air filtration membrane of claim 1, wherein the air filtration membrane is incorporated into a filter for use in HVAC systems.

17. The air filtration membrane of claim 1, wherein the air filtration membrane is incorporated into a filter for use as a window screen.

18. The air filtration membrane of claim 1, wherein the air filtration membrane is incorporated into indoor barrier screens.

19. The air filtration membrane of claim 1, wherein the air filtration membrane is incorporated into indoor modular furniture panels.

20. An air filtration membrane as recited in claim 19, wherein the membrane is configured as a replaceable filter cartridge adapted to slide vertically or horizontally into the modular furniture panel frame and locked into place therein.

21. An air filtration membrane as recited in claim 19, wherein the membrane comprises one or both sides of a lightweight, permeable fabric sleeve configured to cover a modular furniture panel frame.

22. An air filtration membrane as recited in claim 19, wherein the modular furniture panels are adapted and configured to be attached together by way of brackets or clamps.

23. An air filtration membrane as recited in claim 19, wherein the brackets or clamps are magnetized for ease of assembly.

24. A modular furniture panel, comprising:
a structural frame; and
an air filtration membrane operatively associated with the frame, wherein the membrane includes a substrate and a network of electro-spun polymeric nanofibers deposited on the substrate.

25. A modular furniture panel as recited in claim 24, wherein the network has a total polymer weight of 5 to 25% and wherein the membrane has a minimum air filtration efficiency of between MERV 8 and MERV 16.

26. A modular furniture panel as recited in claim 24, wherein the membrane is configured as a filter cartridge adapted for insertion into the frame of the modular furniture panel.

27. A modular furniture panel as recited in claim 24, wherein the membrane is configured as an air permeable sleeve adapted to enclose the frame of the modular furniture panel.

28. A modular furniture panel as recited in claim 24, wherein the frame includes means for connecting plural modular furniture panels together to construct an assembly of furniture panels.
